(19) United States
(12) Patent Application Publication
(10) Pub. No.: US 2014/0094430 A1
(43) Pub. Date: Apr. 3, 2014

(54) HYALURONIC ACID COMPOSITIONS STABILISED AGAINST THE DEGRADING EFFECT OF HEAT OR ENZYMES

(71) Applicants: Carlo Vernasca, Lugano (CH); Andrea Maria Giori, Milano (IT); Stefano Togni, Milano (IT)

(72) Inventors: Carlo Vernasca, Lugano (CH); Andrea Maria Giori, Milano (IT); Stefano Togni, Milano (IT)

(73) Assignee: ALTERGON S.A., Lugano (CH)

(21) Appl. No.: 13/976,113

(22) PCT Filed: Dec. 16, 2011
(86) PCT No.: PCT/EP11/73079

§ 371 Date: Jul. 8, 2013

(30) Foreign Application Priority Data
Dec. 27, 2010 (IT) MI2010A002404

(51) Int. Cl. A61K 31/728

(52) U.S. Cl. CPC A61K 31/728
USPC 514/54; 536/123.1; 536/123

(57) ABSTRACT

The invention relates to the use of an additive to stabilise hyaluronic acid formulated in an aqueous composition against the degrading effect of heat or of enzymes such as hyaluronidase, characterised in that said additive comprises one or more polysaccharides having a molecular weight equal to or greater than 20,000 daltons and solubility in water at least equal to or greater than 1 g/l, the total concentration of said polysaccharide(s) and hyaluronic acid in the composition giving it a viscosity of at least 500 cP, which is particularly suitable for intra-articular, intradermal or intraocular administration to humans or animals.

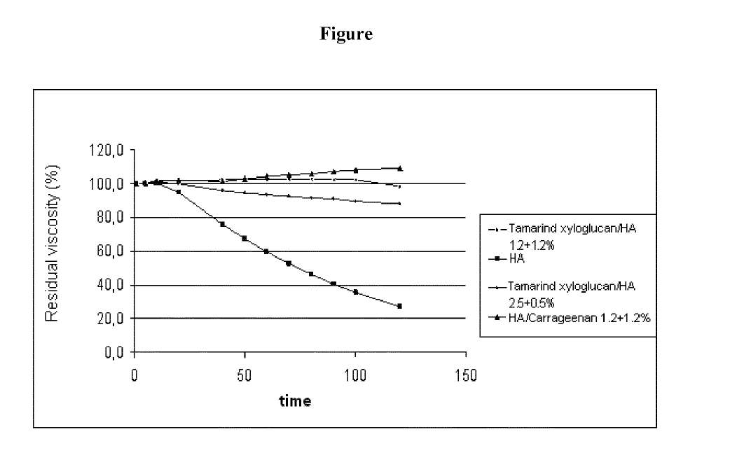

The present invention relates to hyaluronic acid compositions stabilised against the degrading effect of heat or of enzymes such as, primarily, hyaluronidase.

Hyaluronic acid (HA) is a polysaccharide present in all the tissues and biological fluids of vertebrates, particularly in the connective tissue. It belongs to the glycosaminoglycan family and is a linear unbranched polysaccharide formed by the repetition of a disaccharide unit consisting of N-acetyl-D-glucosamine and sodium glucuronate joined by a β1-4 glycosidic bond according to the structure:

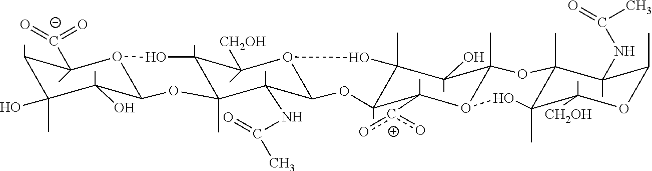

Hyaluronic acid has a mean molecular weight ranging between 10⁴ and 10⁷ daltons. For example, hyaluronic acid with a molecular weight of 7×10⁶ daltons is present in synovial fluid.

Hyaluronic acid and the derivatives thereof, in particular its salts, such as the sodium salt, are widely used for therapeutic purposes. Its high viscosity, rheological properties, biocompatibility and complete biodegradability allow widespread use of hyaluronic acid for medical and dermatological applications. According to the sphere of interest of the present invention, examples of application in the medical field include intraocular injections in ophthalmic surgery, intra-articular injections in orthopaedics to restore the functionality of the synovial fluid, and intradermal fillers for beauty treatments. Other examples of application are to be found in the veterinary field, such as viscosupplementation in the horse.

The physiological and physicochemical properties of hyaluronic acid are associated with its molecular weight. Polymer chains with a high enough weight, like those present in synovial fluid, have a high viscosity which forms the basis of their shock absorption capacity and lubricating property.

An undesirable reduction in molecular weight, such as that due to fragmentation of the polymer chains in case of degradation, lead to loss of the characteristic properties of hyaluronic acid.

The main degradation agents of hyaluronic acid are heat and specific enzymes, especially the enzyme hyaluronidase. Exposure of hyaluronic acid to high temperatures or to the action of hyaluronidase causes fragmentation of the polysaccharide chain, with a reduction in molecular weight and viscosity, and consequently in the characteristic properties of hyaluronic acid.

The instability of hyaluronic acid restricts its use, especially for all applications which require rheological behaviour specific to the polysaccharide with high molecular weight. A set of semi-synthetic derivatives, obtained by crosslinking the hyaluronic acid chains, has been developed for this reason. The crosslinked derivatives are more resistant, in particular to enzymatic degradation by hyaluronidase.

Greater inertia to degradation agents is obtained by structurally modifying hyaluronic acid, by inserting bridging molecules bonded with covalent bonds. In practice, however, a new polymer is obtained in this way which is neither natural nor endogenous, and significantly less biocompatible than hyaluronic acid. This aspect, as well as the potential presence of residues of the crosslinking reaction, is the reason for some undesirable side effects which can occur with these crosslinked derivatives of hyaluronic acid, and therefore restricts their use.

The ability to obtain hyaluronic acid with increased stability, without structural modifications which prejudice its safety and characteristic properties, consequently remains a technical problem which impedes the ideal use of this polysaccharide.

According to the present invention, it has now surprisingly been discovered that adding certain polysaccharide substances to hyaluronic acid improves its stability to degradation agents without introducing any modifications into its primary structure. The present invention therefore proposes the use of an additive to stabilise hyaluronic acid formulated as an aqueous composition against thermal degradation or the action of enzymes such as hyaluronidase, characterised in that said additive comprises one or more polysaccharides having a molecular weight equal to or greater than 20,000 daltons, and water solubility at least equal to or greater than 1 g/l, the total concentration of said polysaccharide(s) and hyaluronic acid in the composition giving it a viscosity of at least 500 cP.

Said viscosity makes the hyaluronic acid-based composition particularly suitable for intra-articular, intradermal or intraocular administration, in both humans and animals.

Further interesting fields of application according to the present invention appear to be otological surgery, tissue engineering and the like.

The invention also relates to aqueous compositions of hyaluronic acid thus stabilised, which are suitable in particular for intra-articular, intradermal or intraocular use.

According to the invention, as demonstrated by the experimental findings described below, hyaluronic acid combined with said polysaccharides acquires improved heat stability and exhibits reduced kinetics of enzymatic degradation.

The polysaccharides that perform this stabilising action are selected from those with a molecular weight exceeding 20,000 daltons, preferably greater than 50,000 daltons, which are water-soluble at a concentration at least equal to or greater than 1 g/l, preferably greater than 2 g/l, giving with hyaluronic acid a final viscous solution with a critical viscosity of not less than 500 cP.

The viscosity of the solutions obtained is measured with a suitable instrument able to operate with solutions having a medium-high viscosity. Suitable instruments are shear rheometers, such as Brookfield rotational viscometers and viscometers, equipped, for example, with rotational cylinder or cone/plate measurement systems. The instrument used in the experimental measurements reported in the subsequent examples of the present description is Brookfield rheometer R/S CPS+, with C-50 spindle.

Said polysaccharide is preferably selected from gellan gum, carrageenans obtained from seaweed such as i-carrageenan, tamarind seed xyloglucan and xanthan gum, as such or mixed together.

By way of example but not of limitation, commercial polysaccharides which can be used to obtain stabilised hyaluronic acid according to the present invention are Phytagel gellan gum, Xilovisc® tamarind seed xyloglucan manufactured by the present Applicant, and Glyloid.

Single polysaccharides with high or commercial-grade purity, or mixtures of two or more polysaccharides, can be used. The polysaccharides, or mixtures thereof, are added to hyaluronic acid in weight ratios of between 1 to 10 and 10 to 1. In stabilised hyaluronic acid, the content of added polysaccharides therefore ranges between 10% and 90% by weight.

Hyaluronic acid suitable for the purposes of the present invention preferably has a molecular weight equal to or greater than 2×10⁵ daltons. For example, a preferred hyaluronic acid has MW =1.8×10⁶ daltons. Another preferred hyaluronic acid has MW=3×10⁵ daltons.

The following composition examples, with the corresponding total dynamic viscosity of the compositions, are given for the purpose of illustration and not of limitation of the present invention.

### COMPOSITION EXAMPLES

### Example 1

| Ingredients | % by w/v |
|---|---|
| Tamarind seed xyloglucan | 1.0 |
| Hyaluronic acid (Mw 1.8 × 10⁶ daltons) | 0.5 |
| Sodium chloride | 0.9 |
| Mono- and dibasic sodium phosphate | q.s. for pH 7.0 ± 0.5 |
| Water for injection | q.s. for 100 ml |

Dynamic viscosity of the composition: 500 cP

### Example 2

| Ingredients | % by w/v |
|---|---|
| Tamarind seed xyloglucan | 0.8 |
| Hyaluronic acid (Mw 1.8 × 10⁶ daltons) | 0.8 |
| Sodium chloride | 0.9 |
| Mono- and dibasic sodium phosphate | q.s. for pH 7.0 ± 0.5 |
| Water for injection | q.s. for 100 ml |

Dynamic viscosity: 750 cP

### Example 3

| Ingredients | % by w/v |
|---|---|
| Tamarind seed xyloglucan | 1.0 |
| Hyaluronic acid (Mw 1.8 × 10⁶ daltons) | 1.0 |
| Sodium chloride | 0.9 |
| Mono- and dibasic sodium phosphate | q.s. for pH 7.0 ± 0.5 |
| Water for injection | q.s. for 100 ml |

Dynamic viscosity: 1000 cP

### Example 4

| Ingredients | % by w/v |
|---|---|
| Tamarind seed xyloglucan | 1.2 |
| Hyaluronic acid (Mw 1.8 × 10⁶ daltons) | 1.2 |
| Sodium chloride | 0.9 |
| Mono- and dibasic sodium phosphate | q.s. for pH 7.0 ± 0.5 |
| Water for injection | q.s. for 100 ml |

Dynamic viscosity: 1500 cP

### Example 5

| Ingredients | % by w/v |
|---|---|
| Tamarind seed xyloglucan | 1.4 |
| Hyaluronic acid (Mw 1.8 × 10⁶ daltons) | 1.4 |
| Sodium chloride | 0.9 |
| Mono- and dibasic sodium phosphate | q.s. for pH 7.0 ± 0.5 |
| Water for injection | q.s. for 100 ml |

Dynamic viscosity: 2300 cP

### Example 6

| Ingredients | % by w/v |
|---|---|
| Tamarind seed xyloglucan | 1.6 |
| Hyaluronic acid (Mw 1.8 × 10⁶ daltons) | 1.6 |
| Sodium chloride | 0.9 |
| Mono- and dibasic sodium phosphate | q.s. for pH 7.0 ± 0.5 |
| Water for injection | q.s. for 100 ml |

Dynamic viscosity: 3000 cP

### Example 7

| Ingredients | % by W/v |
|---|---|
| Carrageenan | 1.6 |
| Hyaluronic acid (Mw 1.8 × 10⁶ daltons) | 1.6 |
| Sodium chloride | 0.9 |
| Mono- and dibasic sodium phosphate | q.s. for pH 7.0 ± 0.5 |
| Water for injection | q.s. for 100 ml |

Dynamic viscosity 2500 cP

### Example 8

| Ingredients | % by W/v |
|---|---|
| Phytagel | 1.4 |
| Hyaluronic acid (Mw 1.8 × 10⁶ daltons) | 1.4 |
| Sodium chloride | 0.9 |
| Mono- and dibasic sodium phosphate | q.s. for pH 7.0 ± 0.5 |
| Water for injection | q.s. for 100 ml |

Dynamic viscosity: 1500 cP

### Example 9

### Intraocular Viscoelastic Composition

| Ingredients | % by W/v |
|---|---|
| Tamarind seed xyloglucan | 2.0 |
| Hyaluronic acid (Mw 1.8 × 10⁶ daltons) | 1.6 |
| Sodium chloride | 0.9 |
| Mono- and dibasic sodium phosphate | q.s. for pH 7.0-7.5 |
| Water for injection | q.s. for 100 ml |

Dynamic viscosity: 3500 cP

### Example 10

| Ingredients | % by W/v |
|---|---|
| Tamarind seed xyloglucan | 1.6 |
| Hyaluronic acid (Mw 300.000 daltons) | 1.6 |
| Sodium chloride | 0.9 |
| Mono- and dibasic sodium phosphate | q.s. for pH 7.0 ± 0.5 |
| Water for injection | q.s. for 100 ml |

Dynamic viscosity: 1000 cP

### Example 11

| Ingredients | % by W/v |
|---|---|
| Tamarind seed xyloglucan | 1.4 |
| Hyaluronic acid (Mw 1.0 × 10⁶ daltons) | 1.4 |
| Sodium chloride | 0.9 |
| Mono- and dibasic sodium phosphate | q.s. for pH 7.0 ± 0.5 |
| Water for injection | q.s. for 100 ml |

Dynamic viscosity: 2000 cP

The following experimental application examples are also described only for the purpose of illustration and not of limitation of the present invention.

The figure in the annexed drawing shows a diagram with special reference to example 16 below, as hereinafter described.

### Example 12

### Heat Stabilisation

The dynamic viscosity of aqueous solutions of hyaluronic acid (HA in the table below) at the concentration of 1.6%, as such and with the addition of the polysaccharides specified in the table, is measured and compared before and after heating at 120° C. for 1 hour. The test is significant because the viscosity is directly correlated with the molecular weight of hyaluronic acid, and its reduction is proportional to the thermal degradation.

The viscosity of the solutions is measured, at the temperature of 25° C., with a Brookfield R/S CPS+ rheometer with a cone/plate system and C-50 spindle, at a speed gradient of 167.6 sec-1.

The values measured (centipoise, cP) are summarised in the table below:

| | Viscosity (cP) | Viscosity (cP) | Viscosity |
|---|---|---|---|
| Solution | before heating | after heating | reduction |
| HA 1.6% | 850 | 520 | −39% |
| 1.6% HA + 1.6% | 2092 | 1850 | −12% |
| Phytagel | | | |
| 1.6% HA + 1.6% | 2948 | 2388 | −19% |
| i-carrageenan | | | |
| 1.6% HA + 1.6% | 1071 | 801 | −25% |
| xanthan gum | | | |

Control of the viscosity reduction due to the addition of the polysaccharide is evident from the values reported.

### Example 13

### Heat Stabilisation in Autoclave

The dynamic viscosity of aqueous solutions of hyaluronic acid (HA in the table) and hyaluronic acid with the addition of polysaccharides is measured and compared before and after autoclave sterilisation. The viscosity is directly correlated with the molecular weight of hyaluronic acid, and its reduction is proportional to the thermal degradation. The sterilisation conditions applied in the autoclave are: temperature:

121+/−1° C., time Fo=13.

The viscosity of the solutions is measured, at the temperature of 25° C., with a Brookfield R/S CPS+rheometer with a cone/plate system and C-50 spindle, at a speed gradient of 167.6 sec-1.

The values measured (cP) are summarised in the table below:

| | Viscosity | Viscosity | |
|---|---|---|---|
| | (cP) before | (cP) after | Viscosity |
| Solution | sterilisation | sterilisation | reduction |
| HA 1.6% | 850 | 587 | −31% |
| 1.6% HA + 1.6% | 2948 | 2447 | −17% |
| i-carrageenan | | | |
| 1.6% HA + 1.6% tamarind | 3381 | 3178 | −6% |
| xyloglucan | | | |

Control of the viscosity reduction due to the addition of the polysaccharide is evident from the values reported.

### Example 14

### Heat Stabilisation in Autoclave

The variation in dynamic viscosity of an aqueous solution of hyaluronic acid (HA in the table) as such is measured, and compared with a solution of hyaluronic acid to which tamarind seed xyloglucan is added, before and after autoclave sterilisation, at different hyaluronic acid concentrations and different tamarind seed xyloglucan concentrations, as specified in the table below.

The sterilisation conditions applied in the autoclave are: temperature: 121+/−1° C., time Fo=13. The viscosity of the solutions is measured, at the temperature of 25° C., with a Brookfield R/S CPS+rheometer with a cone/plate system and C-50 spindle, at a speed gradient of 167.6 sec-1.

The values measured (cP) are summarised in the table below:

| | Viscosity | Viscosity | |
|---|---|---|---|
| | (cP) before | (cP) after | Viscosity |
| Solution | sterilisation | sterilisation | reduction |
| HA 1.6% | 850 | 587 | −31% |
| 1.6% HA + 1.6% tamarind | 3381 | 3178 | −6% |
| xyloglucan | | | |
| 1.4% HA + 1.4% tamarind | 2468 | 2256 | −9% |
| xyloglucan | | | |
| 1.2% HA + 1.2% tamarind | 1811 | 1662 | −8% |
| xyloglucan | | | |
| 1.0% HA + 1.0% tamarind | 1216 | 1048 | −14% |
| xyloglucan | | | |
| 0.4% HA + 2.0% tamarind | 1323 | 1237 | −7% |
| xyloglucan | | | |

### Example 15

### Enzymatic Stabilisation (Hyaluronidase)

An aqueous solution of hyaluronic acid (HA in the table below) is prepared at a concentration of 1.33%, and compared with an aqueous solution of hyaluronic acid with tamarind seed xyloglucan, both at the concentration of 1.33%.

1 g of 0.006% aqueous solution of enzyme hyaluronidase is added to 9.5 g of each solution. The variation in viscosity of the resulting solutions is measured over time, at the temperature of 37° C., using a Brookfield R/S CPS+rheometer with a cone/plate system and C-50 spindle, at a velocity gradient of 167.6 sec-1.

The values measured (cP) are summarised in the table below:

| HA + XYLOGLUCAN | | HA | |
|---|---|---|---|
| time (minutes) | viscosity (cP) | time (minutes) | viscosity (cP) |
| 1 | 1381 | 1 | 408 |
| 5 | 1387 | 5 | 407 |
| 10 | 1403 | 10 | 409 |
| 20 | 1409 | 20 | 387 |
| 40 | 1411 | 40 | 310 |
| 50 | 1413 | 50 | 275 |
| 60 | 1413 | 60 | 243 |
| 70 | 1418 | 70 | 215 |
| 80 | 1417 | 80 | 189 |
| 90 | 1416 | 90 | 165 |
| 100 | 1416 | 100 | 144 |
| 120 | 1357 | 120 | 110 |

### Example 16

### Enzymatic Stabilisation (Hyaluronidase)

A similar variation in viscosity according to the methodology used in the previous example was also measured and compared for the following aqueous solutions of hyaluronic acid (HA):

HA alone at the concentration of 1.2%

1.2% HA with 1.2% tamarind seed xyloglucan

0.5% HA with 2.5% tamarind seed xyloglucan

1.2% HA with 1.2% i-carrageenan

The annexed drawing shows a graph of the residual percentage viscosity values (expressed in cP) obtained up to 120 minutes (time on the x-axis). The stabilisation effect of all the polysaccharides proposed according to the invention is clearly shown.

In conclusion, the experimental findings referred to above demonstrate that adding the polysaccharides according to the invention on to hyaluronic acid produces solutions which can be heated to high temperatures without causing a significant reduction in the molecular weight of hyaluronic acid, as demonstrated by the substantial stabilisation of the viscosity of the solutions.

Similarly, hyaluronic acid combined with the polysaccharides according to the invention is degraded much more slowly by the enzyme hyaluronidase than the corresponding hyaluronic acid without added polysaccharides.

As will be seen from the description and the examples, the aims of the invention are effectively achieved.

1. Method of stabilizing hyaluronic acid formulated in aqueous composition against the degrading effect of heat or of enzymes such as hyaluronidase said method comprising
adding an additive to said hyaluronic in said aqueous composition, wherein, said additive comprises one or more polysaccharides having a molecular weight equal to or greater than 20,000 daltons and solubility in water at least equal to or greater than 1 g/l, the total concentration of said polysaccharide or polysaccharides and hyaluronic acid in the composition giving the composition a viscosity of at least 500 cP; and
stabilizing said hyaluronic acid in said aqueous composition.

2. Method according to claim 1, wherein said composition is suitable for intra-articular, intradermal or intraocular administration, in humans or in animals.

3. Method according to claim 1, wherein said polysaccharide is selected from gellan gum, carrageenans from algae, xyloglucan from tamarind seeds and xanthan gum, and mixtures thereof.

4. Method according to claim 1, wherein said hyaluronic acid has a molecular weight equal to or greater than 2×10⁵ daltons.

5. Method according to claim 1, wherein said hyaluronic acid and said polysaccharide, or mixtures thereof, have a weight ratio in the range between 1:10 and 10:1 respectively.

6. An aqueous composition of hyaluronic acid having a molecular weight equal to or greater than 2×10⁵ daltons, said aqueous composition comprising, as additive for stabilising against the degrading effect of heat or enzymes such as hyaluronidase, at least one polysaccharide having a molecular weight greater than 20,000 daltons and solubility in water at least equal to or greater than 1 g/l, the composition having a viscosity of at least 500 cP.

7. A composition according to claim 6 suitable for intra-articular, intradermal or intraocular administration, in humans or in animals.

8. The composition according to claim 6, wherein said hyaluronic acid and said polysaccharide or mixtures thereof, have a weight ratio in the range between 1:10 and 10:1 respectively.

9. The composition according to claim 6, wherein said polysaccharide is selected from gellan gum, carrageenans from algae, xyloglucan from tamarind seeds and xanthan gum, and mixtures thereof.

10. A polysaccharide having a molecular weight greater than 20,000 daltons and solubility in water at least equal to or greater than 1 g/l, to stabilise against the degrading effect of heat, or enzymes such as hyaluronidase, hyaluronic acid formulated in aqueous composition having, as final composition, a viscosity of at least 500 cP.

11. The polysaccharide according to claim 10, wherein said composition is suitable for intra-articular, intradermal or intraocular administration, in humans or in animals.

12. The polysaccharide according to claim 10, selected from gellan gum, carrageenans from algae, xyloglucan from tamarind seeds and xanthan gum and mixtures thereof.
